(19) United States
(12) Patent Application Publication
(10) Pub. No.: US 2008/0242621 A1
(43) Pub. Date: Oct. 2, 2008

(54) YEAST HYDROLYSATE CONTAINING CYCLO-HIS-PRO AND METHOD OF MAKING AND USING THE SAME

(75) Inventor: HYUNG JOO SUH, SEOUL (KR)

Correspondence Address: KNOBBE MARTENS OLSON & BEAR LLP, 2040 MAIN STREET, FOURTEENTH FLOOR, IRVINE, CA, 92614, US

(73) Assignee: SEROMBIO CO., LTD., GUNPO-SI (KR)

(21) Appl. No.: 12/135,785

(22) Filed: Jun. 9, 2008

Related U.S. Application Data
(63) Continuation of application No. PCT/KR2005/004227, filed on Dec. 9, 2005.

(51) Int. Cl. A61K 38/06; C07K 5/08; A61P 3/10

(52) U.S. Cl. 514/18; 530/331

(57) ABSTRACT

Provided is a method for producing a yeast hydrolysate containing Cyclo-His-Pro (CHP), via an enzyme-substrate reaction by addition of a flavourzyme to a yeast suspension. Herein, the yeast hydrolysate obtained after the enzyme-substrate reaction is filtered or centrifuged to recover a supernatant, and the supernatant may be subjected to (1) drying after ultrafiltration, or (2) drying after activated carbon treatment, or (3) combined treatment of (1) and (2). The method of the present invention enables production of the yeast hydrolysate having a remarkably high content of Cyclo-His-Pro (CHP), as compared to conventional foods. In particular, purification of the thus-resulting yeast hydrolysate can enhance the content of Cyclo-His-Pro (CHP) 10-fold to 85-fold higher than prior to the purification thereof. The yeast hydrolysate containing Cyclo-His-Pro (CHP) of the present invention exerts blood glucose-lowering effects and glucose tolerance-improving effects, simultaneously with an enhancement of insulin sensitivity.

### BACKGROUND

1. Field

The present disclosure relates to a yeast hydrolysate and a method for producing the same. More specifically, the present disclosure relates to a method for producing a yeast hydrolysate having a high content of Cyclo-His-Pro (CHP) and a yeast hydrolysate produced by the same method.

2. Discussion of Related Technology

Peptides in foods play an important role in affecting the nutritional, organoleptic, and functional properties of the foods. Many kinds of peptides present in living organisms exhibit various biochemical activities. Examples of such peptides may include various peptide hormones, neurotransmitters, interleukins, cell growth factors, and bacteriocins. Numerous peptides are formed in the foods, particularly during a fermentation process, and some of them exert a variety of physiological activities. Since 1970s, physiologically active peptides have been focused on applicability and availability thereof as nutraceuticals. In addition, such peptides are known to have various physiological activities such as opioid agonist or antagonist activity, suppression of angiotensin I converting enzyme (ACE), immunomodulatory activity, antibacterial activity, antithrombotic activity, inhibition of lipid oxidation, mineral-binding activity, and the like. That is, milk protein-derived peptides, including casein and lactalbumin, are reported to exert functions such as enhancing phagocytosis or controlling differentiation of lymphocytes. Further, according to recent published research reports, immunomodulatory peptides are produced from soybean proteins and certain peptides, such as HCQRPR and QRPR isolated from enzymatic hydrolysates of proteins containing glycinin as a principle material, not only activate phagocytosis of macrophages but also exhibit immunomodulatory activity such as a defense against bacterial infection and promotion of secretion of tumor necrosis factor (TNF). As the protein-derived active peptides, soybean and milk proteins, e.g. casein-derived proteins have been largely utilized. On the other hand, a great deal of recent interest is focused on yeast that can be produced on an industrial scale. Yeast has been used as a raw material for production of a variety of useful material in the food industry including brewing and bread making since ancient times. Yeast contains about 50% protein, lipid, nucleic acid such as RNA, and various vitamins and minerals. With recent reports of various physiological activities of yeast, yeast extracts have been primarily used to influence individual taste and preference by freely conferring desired flavor to food materials, upon production of processed foodstuffs. In particular, yeast is reported to have the effects of a relaxation response on premenstrual syndrome (PMS) and anti-stress effects. These effects are believed to be due to neurotransmitters produced during fermentation or hydrolysis processing of yeast. The Cyclo-His-Pro (CHP) peptide is also a neurotransmitter and has various physiological activities. Further, it is also reported that large amounts of the Cyclo-His-Pro (CHP) peptide are found in foods, as compared to the human blood.

According to measurement results of CHP content in general foods, it was reported that the CHP is present at a level of 6 pmol/g in cow's milk and 6.58 nmol/g in dried shrimp, which respectively are 5 to 1,500-fold higher than that of the human serum. However, there is yet no attempt in the conventional art in which the neurotransmitter CHP is prepared by the hydrolysis of proteins contained in the foods. Particularly, to the best of our knowledge, there is no case involving incorporation of the CHP into the yeast hydrolysate widely used in food production. The foregoing discussion is to provide background information and does not constitute an admission of prior art.

### SUMMARY

There is a report that a higher content of Cyclo-His-Pro (CHP) is present in foods, as compared to that found in the human blood. However, as discussed hereinbefore, the Cyclo-His-Pro (CHP) is present in very tiny amounts, at a nmol or pmol level, thus making it inadequate for use or application thereof as the material for taking advantage of the physiological activity possessed by the Cyclo-His-Pro (CHP).

One aspect of the invention provides a yeast hydrolysate containing Cyclo-His-Pro (CHP) in an amount of 0.1 to 6.0% (w/w). In one embodiment, the CHP may be in an amount from about 0.06 to about 5.13% (w/w). In another embodiment, the CHP may be in an amount from about 0.12 to about 1.20% (w/w). In still another embodiment, the CHP may be in an amount from about 0.66 to about 5.13% (w/w). In another embodiment, the foregoing yeast hydrolysate, may further comprise a residual amount of at least one protease may be selected from the group consisting of flavourzyme, ficin and autolysin. The foregoing yeast hydrolysate may be produced by a method comprising: providing a suspension of yeast in a solvent; and adding to the suspension at least one protease for inducing an enzyme-substrate reaction, wherein the at least one protease may be selected from the group consisting of flavourzyme, ficin and autolysin.

Another aspect of the invention provides a food item comprising the foregoing yeast hydrolysates.

Another aspect of the invention provides a method for producing the foregoing yeast hydrolysates. The method comprises: providing a suspension of yeast in a solvent; and adding to the suspension at least one protease for inducing an enzyme-substrate reaction, wherein the at least one protease may be selected from the group consisting of flavourzyme, ficin and autolysin. The method may further comprise collecting a supernatant of the enzyme-substrate reaction. The method may further comprise subjecting the supernatant to at least one additional treatment selected from the group consisting of an ultrafiltration and an activated carbon treatment. The method may further comprise drying a resulting product of the additional treatment. In the foregoing method, the yeast in the suspension may be in an amount of 5 to 15% (w/v) relative to the solvent. The at least one protease may be in an amount of 0.5 to 1.5% (w/w) relative to the suspension. The method may further comprise: adjusting pH of the suspension to a range of 6 to 8 at a temperature of 30° C. to 60° C. The method may further comprise: maintaining an adjusted reaction condition for 48 to 72 hours.

A further aspect of the invention provides a pharmaceutical composition comprising: the foregoing yeast hydrolysate or a pharmaceutically acceptable salt thereof; and a pharmaceutically acceptable carrier.

A still further aspect of the invention provides a method of treating diabetes in a patient in need thereof. The method comprising: administering, to a type I diabetic or a type II diabetic, a pharmaceutically effective amount of the foregoing pharmaceutical composition. In the foregoing method, administering may comprise oral administration. Further in the foregoing method, the pharmaceutically effective amount may be about 0.5-0.7 g of the yeast hydrolysate or a pharmaceutically acceptable salt thereof per 1 kg weight of the type I diabetic. The pharmaceutically effective amount may be about 0.5-1 g of the yeast hydrolysate or a pharmaceutically acceptable salt thereof per 1 kg weight of the type II diabetic.

One aspect of the present invention to provide a material having a high content of Cyclo-His-Pro (CHP) suited for taking advantage of physiological activity possessed by Cyclo-His-Pro (CHP), and a method for producing the same.

In accordance with an aspect of the present invention, the above and other benefits can be accomplished by the provision of a method for producing a yeast hydrolysate containing Cyclo-His-Pro (CHP), comprising adding a flavourzyme to a yeast suspension to induce an enzyme-substrate reaction.

Specifically, the method includes preparing 5 to 15% (w/v) of the yeast suspension, adding 0.5 to 1.5% (w/w) of the flavourzyme to the yeast suspension, adjusting a pH of the reaction mixture to a range of pH 6 to 8 at a temperature of 30° C. to 60° C., and subjecting the reaction mixture to the enzyme-substrate reaction for 48 to 72 hours.

Herein, the yeast hydrolysate obtained after the enzyme-substrate reaction is filtered or centrifuged to recover a supernatant, and the resulting supernatant may be dried after ultrafiltration (1) or otherwise may be dried after activated carbon treatment (2). Alternatively, steps 1 and 2 may be carried out concurrently.

In accordance with another aspect of the present invention, there is provided a yeast hydrolysate containing Cyclo-His-Pro (CHP), prepared by the above-mentioned method.

With a method for producing a yeast hydrolysate according to an embodiment of the present invention, it is possible to obtain a yeast hydrolysate having a remarkably higher content of Cyclo-His-Pro (CHP), as compared to conventional foods. In particular, purification of the thus-resulting yeast hydrolysate can enhance the content of Cyclo-His-Pro (CHP) 10-fold to 85-fold higher than prior to the purification thereof.

The yeast hydrolysate containing Cyclo-His-Pro (CHP), provided by the embodiment, particularly exhibits blood glucose-lowering effects and glucose tolerance-improving effects, in conjunction with enhancing insulin sensitivity.

### BRIEF DESCRIPTION OF THE DRAWINGS

The above and other benefits, features and other advantages of the present invention will be more clearly understood from the following detailed description taken in conjunction with the accompanying drawings, in which:

FIG. 1 is a schematic diagram showing a production process of a yeast hydrolysate containing Cyclo-His-Pro (CHP);

FIG. 2 is a graph comparing changes in body weight between normal mice and Type I diabetic mice with and without administration of a yeast hydrolysate containing Cyclo-His-Pro (CHP);

FIG. 3 is a graph comparing changes in blood glucose level between normal mice and Type I diabetic mice with and without administration of a yeast hydrolysate containing Cyclo-His-Pro (CHP);

FIG. 4 is a graph comparing results of an oral glucose tolerance test between normal mice and Type I diabetic mice with and without administration of a yeast hydrolysate containing Cyclo-His-Pro (CHP);

FIG. 5 is a bar graph comparing measurement results of an antidiabetic activity between Type II diabetic (ob/ob) mice with and without oral administration of a yeast hydrolysate containing Cyclo-His-Pro (CHP) for 21 days;

FIG. 6 is a graph comparing results of an oral glucose tolerance test between Type II diabetic (ob/ob) mice with and without administration of a yeast hydrolysate containing Cyclo-His-Pro (CHP); and

FIG. 7 is a bar graph comparing changes in a serum insulin level between Type II diabetic (ob/ob) mice with and without oral administration of a yeast hydrolysate containing Cyclo-His-Pro (CHP) for 21 days under non-fasted conditions and fasted conditions, respectively.

### EMBODIMENTS

Hereinafter, certain embodiments of the present invention will be described.

First, 5 to 15% (w/v) of a yeast suspension was prepared and 0.5 to 1.5% (w/w) of a flavourzyme was added to the yeast suspension. The pH of the resulting mixture was adjusted to a range of 6 to 8 and hydrolyzed at a temperature of 30 to 60° C. for 48 to 72 hours to obtain a yeast hydrolysate. The thus-obtained yeast hydrolysate was filtered or centrifuged and the resulting supernatant was recovered.

The thus-recovered supernatant was subjected to (1) drying after ultrafiltration, or (2) drying after activated carbon treatment, or (3) combined treatment of (1) and (2), thereby preparing a yeast hydrolysate containing Cyclo-His-Pro (CHP).

### EXAMPLES

Now, various features and embodiments of the present invention will be described in more detail with reference to the following examples. These examples are provided only for illustration and should not be construed as limiting the scope and spirit of the present invention.

The following examples consist of selecting a material suited for producing a hydrolysate having a maximum content of Cyclo-His-Pro (CHP) by determination of the CHP content in a yeast hydrolysate; selecting hydrolytic enzymes (hydrolases) suited for producing the yeast hydrolysate; and selecting treatment processes for enhancing the CHP content in the yeast hydrolysate.

As a reference material for analysis of Cyclo-His-Pro (CHP) in the yeast hydrolysate in the following examples, Cyclo-His-Pro (CHP) purchased from Sigma Chemical Co. (St. Louis, Mo., USA) was used. As samples for determining the content of Cyclo-His-Pro (CHP), hydrolysates of pork, shrimp, short-necked clam, cuttlefish, crab and beef were purchased from HaeMa Foods Co., Ltd. (Kyonggi-do, Korea), the yeast hydrolysate was purchased from NEUROTIDE Co., Ltd. (Korea), and the corn gluten hydrolysate was purchased from DAESANG CO., LTD. (Seoul, Korea), respectively.

Among proteases (proteolytic enzymes) used in production of the yeast hydrolysate, neutrase, alcalase, flavourzyme and protamax were purchased from Novo Korea (Seoul, Korea), ficin was purchased from Sigma Chemical Co. (St. Louis, Mo., USA), and bacterial protease was purchased from Amorepacific Corporation (Seoul, Korea). All the other reagents necessary for analysis were first-grade reagents.

The Cyclo-His-Pro (CHP) was analyzed using high performance liquid chromatograph, under the following conditions. 20 μJt, of each sample was loaded to a Hamilton PRP-I RP (pore size-′10 [M, 250 mm×4.1 mm ID, Hamilton Company, Reno, Nev., USA) column and a pre-column Hamilton PRP 10 (pore size: 10 μm, 25 mm×1.3 mm ID, Hamilton Company), and eluted with an eluent (10′-90 (v/v) mixture of an aqueous 0.004 M trifluoroacetic acid solution containing 1-heptanesulfonic acid in a ratio of 0.75 g/L, and acetonitrile) at a flow rate of 0.5 mL/min, followed by determination of the Cyclo-His-Pro (CHP) content in the sample, using a UV detector at a wavelength of 206 nm.

For preparation of the yeast hydrolysate, 8 g of compressed yeast was suspended in 100 mL of distilled water, a pH of the yeast suspension was adjusted to a range of 6 to 8, and 0.5 to 1.5% (w/w) of the protease was added to the suspension, followed by hydrolysis at 50° C. for 48 to 72 hours and centrifugation. The resulting supernatant was dried and used as the yeast hydrolysate.

In order to enhance the content of Cyclo-His-Pro (CHP) contained in the yeast hydrolysate, the yeast hydrolysate was subjected to acid treatment, activated carbon treatment and ultrafiltration. The acid treatment was carried out according to the following procedure. Citric acid was added to a 5% (w/v) yeast hydrolysate solution to adjust a pH thereof to a value of 3.5. The yeast hydrolysate solution was allowed to stand for 30 min and the resulting precipitates were filtered and removed. The filtered solution was neutralized to a pH of 7.0 using NaOH, and dried. The activated carbon treatment was carried out. For this purpose, 1 g of activated carbon was added to 100 mL of a 5% (w/v) yeast hydrolysate solution which was then shaken for 30 min and filtered. The filtrate was removed and 70 mL of 0.3% (v/v) aqueous ammonia was added to the remaining activated carbon which was then shaken for 30 min and filtered. The filtrate was adjusted to a pH of 7.0 and dried. The ultrafiltration treatment was carried out as follows. A 5% (w/v) yeast hydrolysate solution was filtered through a ultrafiltration membrane (PM-IO) having a molecular weight cut-off of less than 10,000, and the filtrates were pooled and dried.

### Example 1

### Selection of Materials for Preparation of Hydrolysates Containing Cyclo-His-Pro (CHP)

Commercially available hydrolysates were purchased and the levels of Cyclo-His-Pro (CHP) therein were determined. The results thus obtained are given in Table 1 below. The yeast hydrolysate exhibited the highest CHP level of 602 μg/g, and the corn gluten hydrolysate and the shrimp hydrolysate exhibited a relatively high CHP level of 23.3 βg/g and 12.3 /zg/g, respectively. These values are higher than those reported by Hilton et al (1992), e.g. 6576 pmol/g in the dried shrimp and 5209 pmol/g in the fish sauce, thus indicating that the shrimp hydrolysate has a CHP content 6-fold higher than that of the dried shrimp, and the yeast hydrolysate has a CHP content about 300-fold higher than that of the dried shrimp, and consequently confirming the applicability thereof as a functional material containing Cyclo-His-Pro (CHP).

TABLE 1

| | CHP level |
|---|---|
| Hydrolysates | (μg/g) |
| Pork | 2.24 |
| Short-necked clam | — |
| Crab | — |
| Shrimp | 12.3 |
| Beef | — |
| Cuttlefish | 1.82 |
| Corn gluten | 23.3 |
| Yeast | 602.3 |

### Example 2

### Selection of Hydrolases Suited for Producing Yeast Hydrolysates

Since a yeast hydrolysate exhibited a high level of Cyclo-His-Pro (CHP) as compared to other hydrolysates in Example 1, different kinds of proteases were used to prepare various yeast hydrolysates and CHP content in the thus-prepared yeast hydrolysates were determined to confirm which kind of protease is suitable to prepare the yeast hydrolysate having a high CHP content.

With reference to Table 2 below, the yeast hydrolysate using a flavourzyme as the protease exhibited the highest CHP level of 674 μg/g and a recovery rate of 16.2% (w/w). The yeast hydrolysate using ficin as the protease exhibited a CHP level of 468 //g/g and the highest recovery rate of 33.8% (w/w). The use of yeast autolysin having a relatively low recovery rate exhibited a CHP level of 603 //g/g, ranking second only to the use of a flavourzyme.

TABLE 2

| | Yield | CHP level |
|---|---|---|
| Proteases | (%) | (μg/g) |
| Neutrase | 21.4 | 502 |
| Alcalase | 21.0 | 286 |
| Bacterial protease | 28.0 | 349 |
| Ficin | 33.8 | 468 |
| Flavourzyme | 16.2 | 674 |
| Protamax | 27.5 | 430 |
| Autolysin | 25.5 | 603 |

### Example 3

### Extracted Volume of Cyclo-His-Pro (CHP) with Varying Contents of Alcohol

Cyclo-His-Pro (CHP) in the yeast hydrolysate is a material having a cyclization structure of histidine and proline and exhibits nonpolarity to some extent. Therefore, an extracted volume of Cyclo-His-Pro (CHP) with varying concentrations of alcohol was determined, taking advantage of the fact that high-molecular weight proteins are sparingly soluble in alcohols, whereas low-molecular weight proteins are readily soluble in alcohols. The results thus obtained are given in Table 3 below. Extraction of Cyclo-His-Pro (CHP) with 95% (v/v) alcohol exhibited a recovery rate of 11.4%, with 0.10% (w/w) of CHP content, thus representing an about 60% increase of the CHP content, as compared to before treatment with alcohol. A decreasing content of alcohol leads to an increase of the yield while decreasing the CHP content.

TABLE 3

| Alcohol concentration | Yield | CHP content |
|---|---|---|
| 95% | 11.4% | 0.10% |
| 80% | 76.8% | 0.06% |
| 60% | 85.0% | 0.01% |

### Example 4

### Effects of Treatment Processes on Enhancement of Content of Cyclo-His-Pro (CHP)

When a yeast hydrolysate was obtained using a flavourzyme, the level of Cyclo-His-Pro (CHP) was about 0.06% (w/w). In order to use the yeast hydrolysate as a functional material for taking advantage of the physiological activity of Cyclo-His-Pro (CHP), it was necessary to increase the CHP content and the yeast hydrolysate was therefore subjected to a purification process.

In order to enhance the CHP content, the yeast hydrolysate was subjected to acid treatment, activated carbon treatment, ultrafiltration treatment and concurrent ultrafiltration and activated carbon treatment, respectively. The CHP content of each dried material thus obtained was measured. The results thus obtained are given in Table 4 below. The ultrafiltration treatment and the acid treatment provided a relatively high yield of 67.4% (w/w) and 51.7% (w/w), respectively. Among single treatments, the ultrafiltration treatment exhibited the highest CHP level of 1.2% (w/w), whereas the activated carbon treatment exhibited a relatively high CHP level of 0.66% (w/w). Among single treatments, the ultrafiltration treatment was a preferred purification process, in terms of the yield and the CHP content.

Combined use of the ultrafiltration treatment and the activated carbon treatment resulted in a sharp decrease of yield, i.e. 4.5% (w/w), but afforded the CHP level of 5.13% (w/w) which is about 85-fold increase, as compared to 0.06% (w/w) corresponding to an initial CHP level of the yeast hydrolysate.

TABLE 4

| Treatment Processes | Yield (%) | CHP content (%) |
|---|---|---|
| Acid treatment | 51.7 | 0.12 |
| Activated carbon treatment | 13.9 | 0.66 |
| Ultrafiltration treatment | 67.4 | 1.20 |
| Ultrafiltration/activated | 4.5 | 5.13 |
| carbon treatment | | |

Based on the results obtained as above, a production process of the yeast hydrolysate containing Cyclo-His-Pro (CHP) is shown in FIG. 1. That is, a 5 to 15% (w/v) yeast suspension was prepared and 0.5 to 1.5% (w/w) of a flavourzyme was added to the yeast suspension. Then, the pH of the yeast suspension was adjusted to a range of 6 to 8 and hydrolyzed at a temperature of 30° C. to 60° C. for 48 to 72 hours to thereby obtain a yeast hydrolysate. The thus-obtained yeast hydrolysate was filtered, or centrifuged, and the resulting supernatant was recovered. The thus-recovered supernatant was subjected to ultrafiltration treatment to obtain a hydrolysate which was then dried, or was subjected to an activated carbon treatment and then drying to prepare a yeast hydrolysate containing Cyclo-His-Pro (CHP).

In accordance with embodiments, it is possible to obtain the yeast hydrolysate containing Cyclo-His-Pro (CHP), particularly the yeast hydrolysate having the CHP level of 0.1 to 6.0% (w/w).

Antidiabetic effects of the thus-obtained yeast hydrolysate containing Cyclo-His-Pro (CHP) were confirmed through the following Experimental Examples.

### Experimental Example 1

### Antidiabetic Effects of Yeast Hydrolysate on Type I Diabetes

In order to examine antidiabetic effects of a yeast hydrolysate containing Cyclo-His-Pro (CHP), the yeast hydrolysate prepared in previous Examples was orally administered to Type I diabetes-induced mice at a dose of 500 mg/kg and 750 mg/kg for 2 weeks, respectively. Each mouse group was designated as STZ-I and STZ-2. On the other hand, Type I diabetes-induced mouse group with no administration of the yeast hydrolysates was designated as STZ-control.

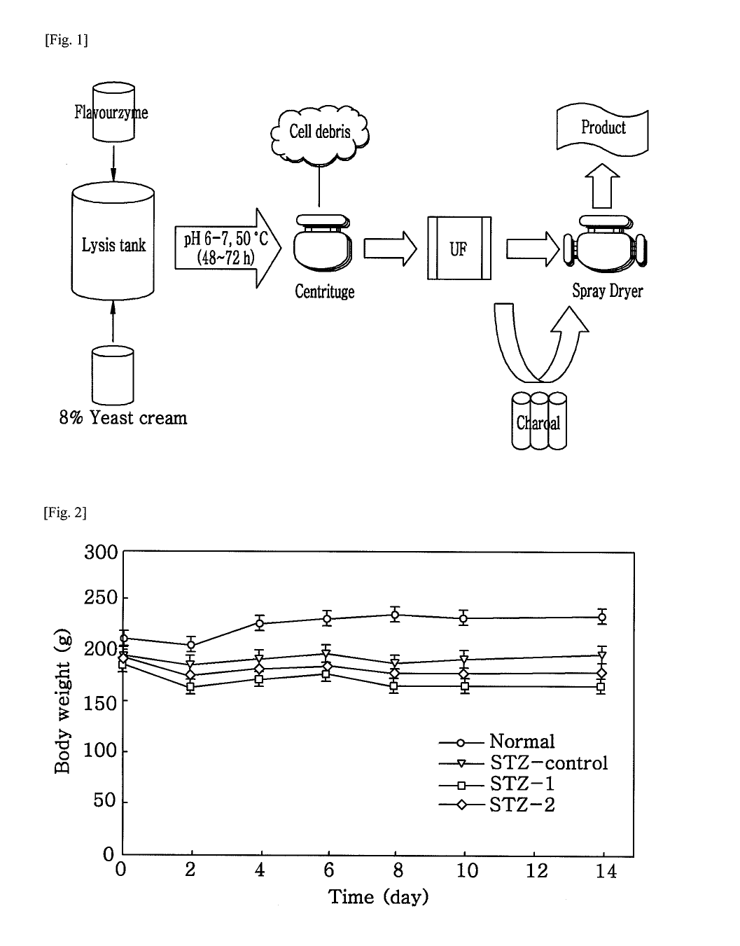

FIG. 2 is a graph comparing changes in body weight between normal mice and Type I diabetic mice with and without administration of the yeast hydrolysate containing Cyclo-His-Pro (CHP).

The normal mouse group showed a gradual increase in body weight during an experimental period of time, whereas the diabetes-induced STZ-control, STZ-I and STZ-2 mouse groups showed no change or slight loss in body weight. Administration of streptozotocin induces diabetes (Type I) due to deficiency of insulin secretion, which in turn leads to disorders of energy metabolism, weight loss and also a difficulty to regain normal body weight unlike alloxan-induced diabetes. Substantially no change or slight loss in body weight was also observed in this experiment.

On the other hand, measurement results of the changes of organ weight of the normal and diabetes-induced mice are given in Table 5 below.

There is a report that the induction of diabetes leads to an increase in liver size (hypertrophy) as compared to that of a normal liver, and the present experiment exhibited a slight increase of liver weight in an oral administration group, as compared to a normal mouse group. Kidney weight was also slightly higher in the oral group, as compared to the normal mouse group. The oral groups (STZ-I and STZ-2) exhibited a relatively low level of the kidney weight, as compared to the STZ-control group. An increased excretion amount due to the induction of diabetes leads to an increased role of the kidney, thereby resulting in renal hypertrophy. As compared to the STZ-control group, a lower predisposition to the renal hypertrophy in the oral groups (STZ-I and STZ-2) suggests that administration of the yeast hydrolysate containing Cyclo-His-Pro (CHP) will provide antidiabetic effects. Further, the spleen also exhibited the similar results.

TABLE 5

| Organs | Normal | STZ-control | STZ-1 | STZ-2 |
|---|---|---|---|---|
| Liver | 4.42 ± 0.20 | 4.92 ± 0.34 | 4.52 ± 0.24 | 0.42 ± 0.18 |
| (g/100 g) | | | | |
| Kidney | 0.48 ± 0.08 | 0.60 ± 0.10 | 0.51 ± 0.10 | 0.54 ± 0.11 |
| (g/100 g) | | | | |
| Spleen | 0.57 ± 0.10 | 0.60 ± 0.09 | 0.58 ± 0.03 | 0.59 ± 0.08 |
| (g/100 g) | | | | |

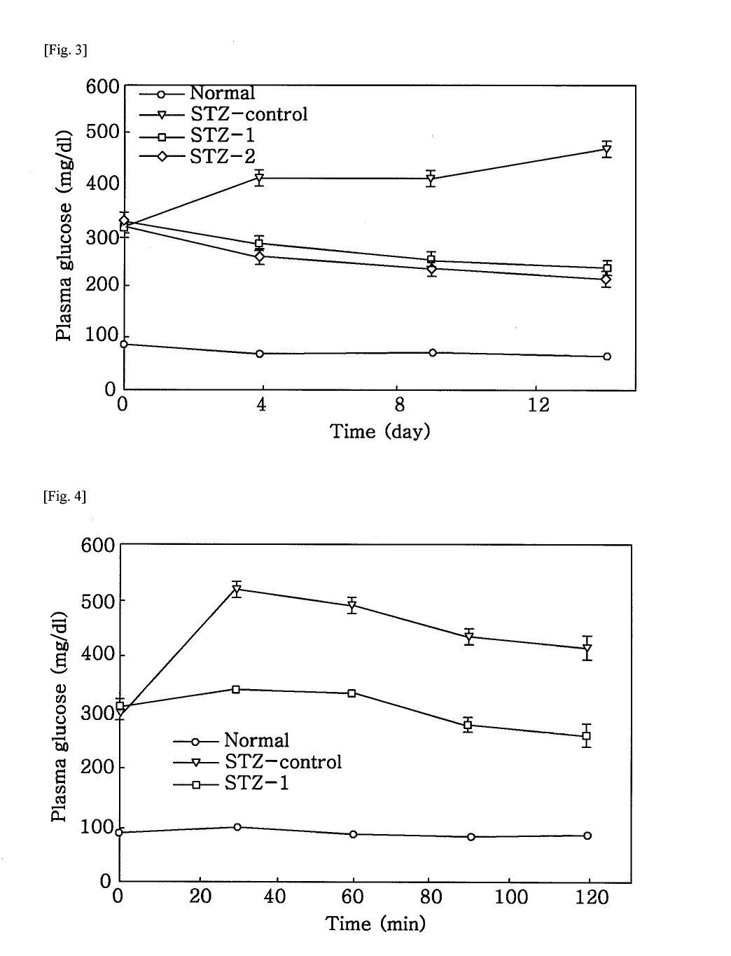

FIG. 3 is a graph comparing changes in a blood glucose level between normal mouse group and Type I diabetic mouse groups with and without administration of a yeast hydrolysate containing Cyclo-His-Pro (CHP).

The normal mouse group exhibited no significant changes in the blood glucose level during the experimental period, whereas the STZ-control group exhibited a continued increase of the blood glucose level throughout the same experiment. On the other hand, each of the STZ-I and STZ-2 groups with oral administration of the yeast hydrolysate containing Cyclo-His-Pro (CHP) exhibited an initial blood glucose level of 312 mg/dL and 325 mg/dL, but the blood glucose level was decreased to 236 mg/dL and 217 mg/dL after a two week oral administration, respectively. From these results, it was demonstrated that the oral administration of the yeast hydrolysate containing Cyclo-His-Pro (CHP) has blood glucose-lowering effects.

Glucose absorbed into the body is burned in peripheral tissues, or stimulates pancreatic beta cells to secrete insulin, thereby controlling the blood glucose level through the feedback action. As such, the body's ability to normally metabolize glucose is called glucose tolerance. Hyperglycemia may be due to a decrease of the glucose tolerance. Therefore, in order to verify blood glucose control effects of functional foods, it will be necessary to perform a glucose tolerance test on individuals.

FIG. 4 is a graph comparing results of the oral glucose tolerance test between normal mice and Type I diabetic mice with and without administration of a yeast hydrolysate containing Cyclo-His-Pro (CHP).

For the oral glucose tolerance test (OGTT), Type I diabetes-induced mice fasted for 12 hours, and the fasting blood glucose level thereof was determined. Thereafter, 750 mg/kg of the yeast hydrolysate containing Cyclo-His-Pro (CHP) was orally administered to the animals to which were then administered 2 g/kg of 30% (w/v) glucose 30 min later, and changes over time in the blood glucose level were determined (diabetic mice of the STZ-2 group). Normal mice and the STZ-control diabetic mice were also subjected to the same experiment, except that the yeast hydrolysate containing Cyclo-His-Pro (CHP) was not administered to the animals. 30 min after administration of glucose, all of the normal mouse group, the STZ-control group and the STZ-2 group exhibited increases in the blood glucose level. It was confirmed that normal mice showed the glucose tolerance, and fluctuation in the blood glucose level in response to glucose administration was insignificant. On the other hand, the diabetes-induced STZ-control group and STZ-2 group were found to exhibit severe fluctuation in the blood glucose level. In particular, the STZ-control group exhibited more significant changes in the blood glucose level, as compared to the STZ-2 group, thus reflecting that the glucose tolerance is significantly lowered in the STZ-control group. On the other hand, the STZ-2 group exhibited relatively little changes in the blood glucose level, as compared to the STZ-control group, and it is therefore considered that the STZ-2 group has the glucose tolerance to some extent.

From the above-mentioned results, it can be seen that oral administration of the yeast hydrolysate containing Cyclo-His-Pro (CHP) provides blood glucose-lowering effects and glucose tolerance-improving effects. Consequently, it will be possible to utilize the yeast hydrolysate containing Cyclo-His-Pro (CHP) as a functional food material which is capable of lowering the blood glucose level.

### Experimental Example 2

### Antidiabetic Effects of Yeast Hydrolysate on Type II Diabetes

In order to examine antidiabetic effects of a yeast hydrolysate containing Cyclo-His-Pro (CHP), the yeast hydrolysate prepared in previous Examples was orally administered to Type II diabetes-induced mice at a dose of 0.5 g/kg and 1.0 g/kg for 3 weeks, respectively. Each mouse group was designated as YH-I and YH-2. On the other hand, Type II diabetes-induced mouse group with no administration of the yeast hydrolysates was designated as a control group.

Body weight gain and food efficiency ratio (FER), and changes of organ weight in the ob/ob mice having genetically induced diabetes (Type II) with and without oral administration of the yeast hydrolysate containing Cyclo-His-Pro (CHP) at a dose of 0.5 g/kg and 1.0 g/kg for 3 weeks are given in Tables 6 and 7, respectively.

The oral administration of the yeast hydrolysate containing Cyclo-His-Pro (CHP) at a dose of 1.0 g/kg exhibited the body weight gain of 0.378 g, thus representing a decrease in the body weight gain, as compared to the control group. In particular, the body weight gain was decreased with an increasing content of the CHP, and therefore it is believed that such body weight loss is due to the physiological activity possessed by the Cyclo-His-Pro (CHP).

TABLE 6

| | Food intake | Body weight gain | |
|---|---|---|---|
| Groups | (g/day) | (g/day) | FER* |
| Control | 5.47 ± 0.11 | 0.551 ± 0.011 | 0.101 ± 0.015 |
| YH-1 (0.5 g/kg) | 5.35 ± 0.07 | 0.479 ± 0.055 | 0.090 ± 0.010 |
| YH-2 (1.0 g/kg) | 4.97 ± 0.26 | 0.378 ± 0.045 | 0.076 ± 0.011 |

*FER = Body weight gain/Food intake

TABLE 7

| | Liver | Kidney | Spleen | Perirenal fat | Epididymal |
|---|---|---|---|---|---|
| Groups | (g/100 g) | (g/100 g) | (g/100 g) | (g/100 g) | fat (g/100 g) |
| Control | 8.19 ± 0.48 | 0.68 ± 0.03 | 0.17 ± 0.01 | 3.38 ± 0.08 | 6.68 ± 0.28 |
| YH-1 | 7.23 ± 0.14 | 0.67 ± 0.02 | 0.16 ± 0.01 | 2.90 ± 0.07 | 6.39 ± 0.25 |
| (0.5 g/kg) | | | | | |
| YH-2 | 7.11 ± 0.75 | 0.72 ± 0.01 | 0.16 ± 0.03 | 2.56 ± 0.27 | 6.74 ± 0.13 |
| (1.0 g/kg) | | | | | |

Effects of oral administration of the yeast hydrolysate containing Cyclo-His-Pro (CHP) on changes of lipid metabolism in the ob/ob mice with genetically-induced diabetes are given in Table 8 below.

YH-I and YH-2 groups exhibited a low value in triglyceride (TG) and total cholesterol, as compared to the control group. In addition, YH-I and YH-2 groups exhibited a significantly high value (pθ0.05) of high density lipoprotein-cholesterol (HDL-chol), as compared to the control group. Further, a value of low density lipoprotein-cholesterol (LDL-chol) was also significantly low (p<0.05) in YH-I and YH-2 groups, as compared to the control group. An atherogenic index, corresponding to the numerical expression of atherogenic risk factors, can be calculated as follows: (Total cholesterol-HDL cholesterol)/HDL cholesterol. The atherogenic index was significantly low (pθ0.05) in the oral administration groups, i.e. YH-I and YH-2 groups, as compared to the control group.

From the above results, it can be seen that the oral administration of the yeast hydrolysate having a high content of Cyclo-His-Pro (CHP) exhibits lipid profile-improving effects in the diabetes-induced ob/ob mice.

TABLE 8

| | TG | TC | HDL-chol | LDL-chol | Atherogenic |
|---|---|---|---|---|---|
| Groups | (mg/dL) | (mg/dL) | (mg/dL) | (mg/dL) | index |
| Control | 73.6 ± 10.7 | 179.8 ± 10.9 | 26.5 ± 5.0 | 150.6 ± 13.9 | 6.5 ± 1.5 |
| YH-1 | 66.0 ± 11.5 | 167.7 ± 7.8 | 167.7 ± 7.8 | 87.4 ± 15.1 | 1.5 ± 0.3 |
| (0.5 g/kg) | | | | | |
| YH-2 | 68.0 ± 19.1 | 68.0 ± 19.1 | 64.0 ± 8.5 | 98.4 ± 6.5 | 1.8 ± 0.4 |
| (1.0 g/kg) | | | | | |

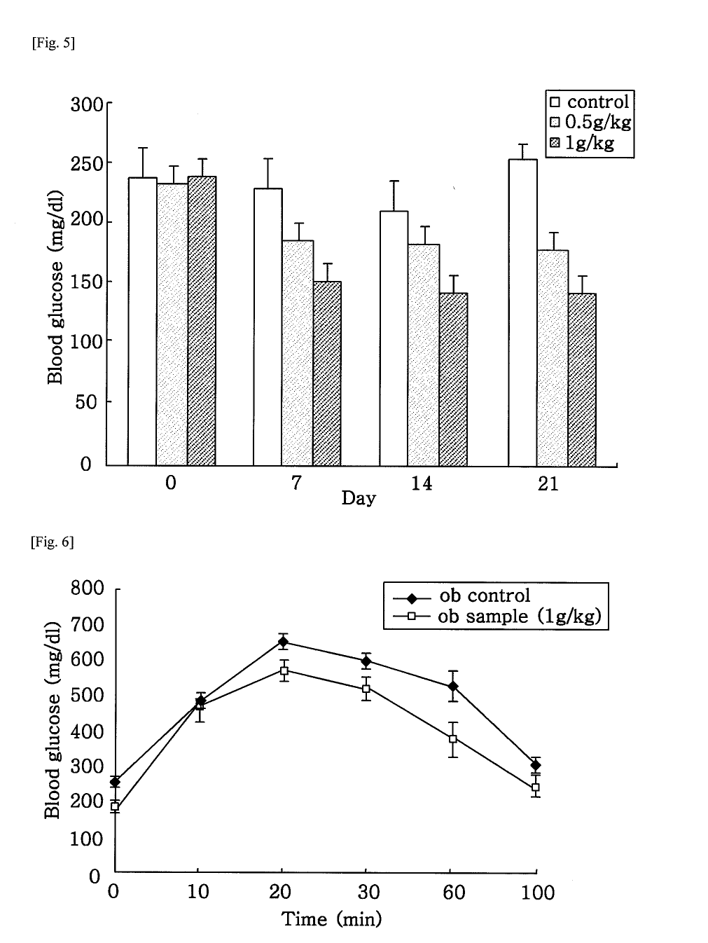

FIG. 5 is a bar graph comparing measurement results of an antidiabetic activity between Type II diabetic (ob/ob) mice with and without oral administration of a yeast hydrolysate containing Cyclo-His-Pro (CHP) for 21 days.

Upon determining the antidiabetic activity after oral administration of the yeast hydrolysate to Type II diabetes-induced ob/ob mice for 21 days, the oral administration groups (YH-I and YH-2) exhibited a significant decrease (pθ0.05) of the blood glucose level 7 days after oral administration, as compared to the control group. There was also a significant difference (p<0.05) in the blood glucose level between the oral groups, i.e. YH-I (0.5 g/kg) and YH-2 (1.0 g/kg). Changes in the blood glucose level at time points of day 14 and day 21 after 7 days did not show a big difference, as compared to the 7-day oral administration group. Therefore, it can be seen that blood glucose-lowering effects by the oral administration of the yeast hydrolysate containing Cyclo-His-Pro (CHP) are expressed prior to 7 days, from the fact that the blood glucose level is in the steady state after 7 days.

For the same purpose as in Type I diabetes-induced mice, the oral glucose tolerance test (OGTT) was carried out, except that Type II diabetic mice (ob control), and the YH-2 group with oral administration of the yeast hydrolysate containing Cyclo-His-Pro (CHP) at a dose of 1.0 g/kg were included in the test.

FIG. 6 is a graph comparing results of the oral glucose tolerance test between Type II diabetic (ob/ob) mice with and without administration of a yeast hydrolysate containing Cyclo-His-Pro (CHP).

As shown in FIG. 6, both of the control group and YH-2 (1.0 g/kg) group exhibited increases in the blood glucose level 20 min after administration of glucose.

The range of changes in the blood glucose level was relatively low in YH-2 group, as compared to the diabetes-induced control group. Based on these results, it can be seen that the glucose tolerance of the group consuming the yeast hydrolysate containing Cyclo-His-Pro (CHP) is relatively high.

From the above-mentioned results, it can be seen that oral administration of the yeast hydrolysate containing Cyclo-His-Pro (CHP) provides blood glucose-lowering effects and glucose tolerance-improving effects.

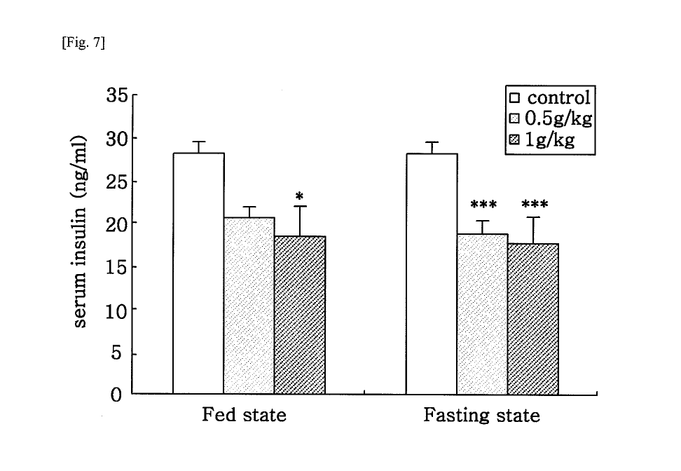

FIG. 7 is a bar graph comparing changes in a serum insulin level between Type II diabetic (ob/ob) mice with and without oral administration of a yeast hydrolysate containing Cyclo-His-Pro (CHP) for 21 days under non-fasted conditions and fasted conditions, respectively.

As shown in FIG. 7, the oral administration group of the yeast hydrolysate containing Cyclo-His-Pro (CHP) exhibited a low insulin content, as compared to the control group. Therefore, it can be seen that the oral administration group has high insulin sensitivity.

Taken altogether, it can be seen that the yeast hydrolysate containing Cyclo-His-Pro (CHP) exerts blood glucose-lowering effects and glucose tolerance-improving effects, in conjunction with enhancement of insulin ′i sensitivity. Consequently, it will be possible to utilize the yeast hydrolysate containing Cyclo-His-Pro (CHP) as a functional food material which is capable of lowering the blood glucose level.

The present invention enables production of a yeast hydrolysate having a remarkably high content of Cyclo-His-Pro (CHP), as compared to conventional foods. In particular, purification of the thus-resulting yeast hydrolysate can enhance the content of Cyclo-His-Pro (CHP) 10-fold to 85-fold higher than prior to the purification thereof. Therefore, the yeast hydrolysate can be very usefully employed as a functional food material.

For example, it will be possible to utilize the yeast hydrolysate as antidiabetic functional food materials which are capable of lowering the blood glucose level.

Although embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A yeast hydrolysate containing Cyclo-His-Pro (CHP) in an amount of 0.1 to 6.0% (w/w).

2. The yeast hydrolysate of claim 1, wherein the CHP is in an amount from about 0.06 to about 5.13% (w/w).

3. The yeast hydrolysate of claim 1, wherein the CHP is in an amount from about 0.12 to about 1.20% (w/w).

4. The yeast hydrolysate of claim 1, wherein the CHP is in an amount from about 0.66 to about 5.13% (w/w).

5. The yeast hydrolysate of claim 1, further comprising a residual amount of at least one protease is selected from the group consisting of flavourzyme, ficin and autolysin.

6. The yeast hydrolysate of claim 1, wherein the yeast hydrolysate is produced by a method comprising:
providing a suspension of yeast in a solvent; and
adding to the suspension at least one protease for inducing an enzyme-substrate reaction, wherein the at least one protease is selected from the group consisting of flavourzyme, ficin and autolysin.

7. A food item comprising the yeast hydrolysate of claim 1.

8. A method for producing the yeast hydrolysate of claim 1, the method comprising:
providing a suspension of yeast in a solvent; and
adding to the suspension at least one protease for inducing an enzyme-substrate reaction, wherein the at least one protease is selected from the group consisting of flavourzyme, ficin and autolysin.

9. The method of claim 8, further comprising collecting a supernatant of the enzyme-substrate reaction.

10. The method of claim 9, further comprising subjecting the supernatant to at least one additional treatment selected from the group consisting of an ultrafiltration and an activated carbon treatment.

11. The method of claim 10, further comprising drying a resulting product of the additional treatment.

12. The method of claim 8, wherein the yeast in the suspension is in an amount of 5 to 15% (w/v) relative to the solvent.

13. The method of claim 8, wherein the at least one protease is in an amount of 0.5 to 1.5% (w/w) relative to the suspension.

14. The method of claim 8, further comprising: adjusting pH of the suspension to a range of 6 to 8 at a temperature of 30° C. to 60° C.

15. The method of claim 14, further comprising: maintaining an adjusted reaction condition for 48 to 72 hours.

16. A pharmaceutical composition comprising:
the yeast hydrolysate of claim 1 or a pharmaceutically acceptable salt thereof; and
a pharmaceutically acceptable carrier.

17. A method of treating diabetes in a patient in need thereof, the method comprising:
administering, to a type I diabetic or a type II diabetic, a pharmaceutically effective amount of the pharmaceutical composition of claim 16.

18. The method of claim 17, wherein administering comprises oral administration.

19. The method of claim 17, wherein the pharmaceutically effective amount is about 0.5-0.7 g of the yeast hydrolysate or a pharmaceutically acceptable salt thereof per 1 kg weight of the type I diabetic.

20. The method of claim 17, wherein the pharmaceutically effective amount is about 0.5-1 g of the yeast hydrolysate or a pharmaceutically acceptable salt thereof per 1 kg weight of the type II diabetic.
